(19) United States
(12) Patent Application Publication
(10) Pub. No.: US 2018/0193249 A1
(43) Pub. Date: Jul. 12, 2018

(54) Use of Bacteriorhodopsin as a Cosmetic and/or Therapeutic Agent in the Comprehensive Treatment of Dermatoses

(71) Applicants: Elizaveta S. GRISHKOVA, Moscow (RU); Alexandre SHNYRA, (US)

(72) Inventor: Elizaveta S. GRISHKOVA, Moscow (RU)

(21) Appl. No.: 15/911,601

(22) Filed: Mar. 5, 2018

Related U.S. Application Data
(63) Continuation of application No. PCT/RU2015/000560, filed on Sep. 3, 2015.

(51) Int. Cl. A61K 8/96; A61Q 19/00; A61K 38/02; A61K 35/74; A61P 17/06; A61P 17/04

(52) U.S. Cl. CPC A61K 8/96; A61Q 19/00; A61P 17/04; A61K 35/74; A61P 17/06; A61K 38/02

(57) ABSTRACT

The invention relates to the field of medicine, in particular to cosmetology, and concerns the development of therapeutic and cosmetic products used in cosmetology and dermatology. The technical result achieved in the implementation of the claimed invention consists in the creation of a new preparation possessing the properties of a medical-cosmetic agent, by using bacteriorhodopsin as an active component of a cosmetic skin care product in the norm and/or a therapeutic agent in the complex treatment of dermatoses by disclosing new specific biological features of bacteriorhodopsin of halophilic bacteria Halobacterium salinarum VKPM V-11850. This technical result is achieved by application an aqueous bacteriorhodopsin solution of the halophilic bacteria Halobacterium salinarum strain VKPM V-11850 containing bacteriorhodopsin at a concentration of 0.24%-0.75% as a cosmetic agent for maintaining the normal functioning of the facial and body skin and/or a remedy for complex treatment of dermatoses.

### RELATED APPLICATIONS

This Application is a Continuation application of International Application PCT/RU2015/000560, filed on Sep. 3, 2015, which is incorporated herein by reference in its entirety.

### FIELD OF THE INVENTION

The invention relates to the field of medicine, in particular to cosmetology, and concerns the development of therapeutic and cosmetic products used in cosmetology and dermatology.

### BACKGROUND OF THE INVENTION

In modern cosmetology, three main branches are distinguished: aesthetic, medical and treatment-and-prophylactic cosmetology. Aesthetic cosmetology deals with a wide range of issues related to maintaining the normal functioning of the skin and its appendages; medical cosmetology treats skin and its appendages at the medical level; and the treatment-and-prophylactic cosmetology, the so-called cosmeceuticals, is aimed at eliminating and preventing dermatological disorders and dehydration of the skin, slowing down its aging processes; it includes active components of predominantly natural origin, which can suppress inflammatory processes, remove free radicals and reduce their formation, improve blood circulation, influence metabolic and regenerative processes in the skin.

To date, cosmeceuticals are of great interest due to the fact that cosmetics of this class work at the cellular level, i.e. they can have a positive effect on the biochemical processes in the body. The composition of such cosmetics is non-toxic and safe for the skin and the body as a whole, as it contains natural bioactive components obtained through high technologies and scientific development in the field of nutritiology (cell nutrition). The production is used by people suffering from diseases such as psoriasis, eczema, etc., since it is 94-98% hypoallergenic. Cosmeceuticals have a prolonged positive effect on the skin, i.e. the result achieved by rejuvenating the skin is maintained for a long time after the abolition of these products, since they work in five layers of the epidermis and two layers of the dermis to the hypodermis.

Cosmeceutical agents include preparations containing, for example:

Fruit acids (AHA)—soft peelings smoothing the skin;

Vitamin E—a complex antioxidant, a “trap” for free radicals that has an anti-inflammatory effect and protects the skin from premature aging, preventing the formation of wrinkles;

Vitamin D—has a noticeable activity in the areas of cell proliferation and differentiation;

Vitamin A and sea buckthorn oil—promote the acceleration of epithelialization;

Hyaluronic acid—a natural moisturizer that protects the skin from drying out;

Ceramides—components that act as a barrier in the stratum corneum and serve to restore the natural state of the skin;

Sun protection factors—UV filters preventing skin burns by UV-rays.

Medicinal plants such as Matricaria, Rose, Aloe, Panax, Hypericum, Urtica, Parsley, Sorbus, Cucumber—suppress the growth of pathogenic microorganisms;

A suspension of biologically active components of bifidobacteria and halobacteria—stimulates metabolic processes in the skin, promotes the regeneration of epidermal cells.

From the point of view of production and obtaining of cosmetic products for skin care technology, the suspensions of biologically active components of bifidobacteria and halobacteria are the most attractive. The seasonality factor is excluded in the process of obtaining these suspensions, in contradistinction to the case with medicinal plants. In addition, the suspensions themselves already contain biologically active compounds synthesized by microorganisms during their growth. All these biologically active substances were synthesized and balanced by nature itself; therefore, they are not alien to the body and are perfectly assimilated. A disadvantage of such suspensions is a limited range of application; more precisely, they are used only in the complex treatment of skin problems.

From the state of the art, it is also known to use a suspension of active components based on halobacterial biomass. Based on the strain of halophilic bacteria Halobacterium halobium 353 Pushchinsky, a preparation was produced that is a lyophilically dried powder of halobacterial biomass of the non-pathogenic strain Halobacterium Halobium 353P called “Baxin”, disclosed in RU 2109515 patent published on 27 Apr. 1998. The “Baxin” powder is used as a biologically active additive to food—nutraceutical.

### SUMMARY OF THE INVENTION

The task for solving by the claimed invention is to expand the arsenal of cosmetic products used as cosmetic skin care products in the complex treatment of dermatoses such as psoriasis, eczema, neurodermatitis, and to maintain the normal functioning of the facial and body skin.

The technical result achieved in the implementation of the claimed invention consists in the creation of a new preparation possessing the properties of a medical-cosmetic agent, by using bacteriorhodopsin as an active component of a cosmetic skin care product in the norm and/or a therapeutic agent in the complex treatment of dermatoses by disclosing new specific biological features of bacteriorhodopsin of halophilic bacteria Halobacterium salinarum VKPM V-11850.

This technical result is achieved by using an aqueous solution of bacteriorhodopsin of the strain of halophilic bacteria Halobacterium salinarum VKPM V-11850 containing bacteriorhodopsin at a concentration of 0.24%-0.75% as a cosmetic agent for maintaining the normal functioning of the facial and body skin and/or a remedy for complex treatment of dermatoses.

Bacteriorhodopsin is a retinal-containing protein of purple membranes of Halobacterium salinarum. Retinal is a derivative of vitamin A

Strains of halophilic bacteria Halobacterium salinarum are natural inhabitants of salt lakes, where the concentration of NaCl can reach 4M, which is 6 times higher than in seawater. Bacteriorhodopsin was previously used as a plant growth stimulant and, in this case, bacterinhodopsin retinal was used as a physiological regulator of vital activity of plant cells. However, as it turned out during the research, bacteriorhodopsin favorably affects not only the growth and development of plant cells, but also the growth and normal functioning of human skin cells. This allows the use of bacteriorhodopsin for a new purpose, namely, to obtain pharmaceuticals that have the necessary properties to maintain the normal functioning of the facial and body skin or in the complex treatment of skin problems.

Bacteria of the Halobacterium salinarum genus do not possess zoopathogenic or phytopathogenic properties and do not pose danger to humans, so working with them does not require special precautions. These findings are confirmed by the research of Thomas Cavalier-Smith, published in his article “Cavalier-Smith T. The neomuran origin of archaebacteria, the negibacterial root of the universal tree and bacterial megaclassification. Int. J. of Syst. And Envir. Microb. 2002, 52, 7-76”.

### DETAILED DESCRIPTION OF THE PREFERRED EMBODIMENTS

The bacteriorhodopsin preparation is obtained as follows.

The strain of halophilic bacteria Halobacterium salinarum VKPM V-11850 is grown in 750 ml flasks in a volume of a growing medium of 300 ml under fluorescent lighting until the stationary growth stage on a circular shaker at 37° C. and shaking 100 rpm. For cultivation, the medium of the following composition is used (mass %): peptone—1, yeast extract—0.5, NaCl—25, MgSO₄—2, KCl—0.2, sodium citrate—0.3, glycerin—0.1, CaCl₂—0.02, water—the rest, pH of the environment—7.2-7.4.

After 6 days, the precipitate is separated by centrifugation at 7000 g for 10 minutes and the obtained biomass is weighed. Subsequently, distilled water in a volume of 300 ml is added to the precipitate (20 g) and incubated with stirring at room temperature for 16 hours.

After centrifugation at 7000 g for 10 min and 4° C., the precipitate is discarded and the resulting supernatant is centrifuged at 50,000 g for 40 min and 4° C. The supernatant is discarded, distilled water is poured into the resulting precipitate in the same volume, the precipitate is suspended, and the resulting bacterial lysate suspension is centrifuged at 50,000 g for 40 minutes and 4° C. Such manipulation of suspending the precipitate in water followed by high-speed centrifugation is repeated until the ratio of the optical density of the solution at 280 nm to the optical density of the solution at a wavelength of 570 nm (D280\D570) is less than 2.5 in the suspended precipitate after the next centrifugation. After that, the supernatant is diluted with distilled water to a concentration of bacteriorhodopsin of 2.4 mg/ml, 7.2 mg/ml. The amount of bacteriorhodopsin is determined at a wavelength of 570 nm, and the concentration is calculated by the following formula:

C=D·Mr·Vsol·Vcuv/Ebr·Vsample,

where:

D is the optical density of the solution at a wavelength of 570 nm;

Mr is the molecular weight of bacteriorhodopsin (26,700);

Vsol is the total volume of the bacteriorhodopsin solution;

Vcuv is the volume of a solution of bacteriorhodopsin in a spectrophotometric cuvette;

Ebr is the molar absorption coefficient of bacteriorhodopsin (63000 M-1cm-1);

Vsample is the volume of a sample of bacteriorhodopsin in a spectrophotometric cuvette.

### EXAMPLE 1

The use of a cosmetic agent to maintain the normal functioning of the facial and body skin.

An aqueous solution of bacteriorhodopsin is used at a concentration of 2.4 mg/ml (0.24%).

Patients aged 23-47 applied the product to dry clean skin of the face and body. The proposed cosmetic product is applied once and daily. The effect of the application is achieved on days 2-3. In the future, after reaching the desired effect, the proposed remedy is used at the discretion of the patient. Some patients treated the face with a tonic before applying the bacteriorhodopsin solution.

All patients noted that the proposed remedy for facial and body skin care provides an improvement in the appearance of the skin. The skin acquires smoothness, softness, elasticity, wrinkles are smoothed, the skin color acquires a fresh, healthy appearance. No adverse effects were observed for the entire duration of the application of the proposed agent.

### EXAMPLE 2

The use of a cosmetic agent for psoriasis treatment.

An aqueous solution of bacteriorhodopsin is used at a concentration of 2.4 mg/ml (0.24%).

Patients aged 33-57 applied the product to the affected area of the skin. The proposed cosmetic product is applied once and daily. The effect of the application is achieved on days 4-7. Later, after reaching the desired effect, recurrence of the disease was not observed.

All patients noted that the proposed remedy eliminates the affected areas on the skin and provides an improvement in the appearance of the skin. The skin acquires a natural healthy appearance. No adverse effects were observed for the entire duration of the application of the proposed agent. No side effects were observed during the application of the recommended agent.

### EXAMPLE 3

The use of a cosmetic agent for neurodermatitis treatment.

An aqueous solution of bacteriorhodopsin is used at a concentration of 2.4 mg/ml (0.24%) or 7.5 mg/ml (0.75%).

Patients aged 30-67 applied the product to the affected area of the skin. The proposed cosmetic product was applied daily in the morning and in the evening. In the case of exacerbation of the neurodermatitis within a period not exceeding 4 months, a solution of bacteriorhodopsin at a concentration of 2.4 mg/ml (0.24%) was used. In the first day (up to 5 days) of the use of the product, a decrease and disappearance of the itching, a decrease of affected areas and a decrease in the intensity of inflammation of the cutaneous lesions were observed. The maximum effect of the proposed solution was observed in 3 weeks. No side effects were observed during the application of the recommended agent.

During the course of neurodermatitis in chronic and prolonged stages, a bacteriorhodopsin solution of 7.5 mg/ml (0.75%) is used. In this case, the proposed cosmetic product was also applied daily in the morning and in the evening. The clinical picture of the effect of using the recommended preparation was the same as described for a concentration of 2.4 mg/ml (0.24%). No side effects were observed during the application of the recommended agent.

### EXAMPLE 4

The use of a cosmetic agent for atopic dermatitis treatment.

An aqueous solution of bacteriorhodopsin is used at a concentration of 2.4 mg/ml (0.24%) and 7.5 mg/ml (0.75%) was used.

Patients aged 18-23 applied the product to the affected area of the skin. The proposed cosmetic remedy was applied daily in the morning and in the evening. In case of exacerbation of atopic dermatitis within a period not exceeding 4 months, a solution of bacteriorhodopsin at a concentration of 2.4 mg/ml (0.24%) was used. In the first day (up to 5 days) of the use, a decrease and disappearance of the itching, a decrease of affected areas and a decrease in the intensity of inflammation of the cutaneous lesions were observed. The maximum effect of the proposed solution was observed in 3 weeks. No side effects were observed during the application of the recommended agent. Some patients used an additional moisturizer and applied it after a solution of bacteriorhodopsin.

During the course of atopic dermatitis in chronic and prolonged stages, a solution of bacteriorhodopsin at a concentration of 7.5 mg/ml (0.75%) is used. In this case, the proposed cosmetic product was also applied daily in the morning and in the evening. The clinical picture of the effect of using the recommended preparation was the same as that described for a concentration of 2.4 mg/ml (0.24%). No side effects were observed during the application of the recommended agent.

What is claimed is:

1. An agent for skin treatment, the agent being an aqueous solution comprising bacteriorhodopsin from a halophilic bacterium Halobacterium salinarum VKPM V-11850 at a concentration of from 0.24 to 0.75% (wt/vol).

2. The agent of claim 1, wherein the agent is a cosmetic agent.

3. The agent of claim 1, wherein the agent is a therapeutic agent.

4. The agent of claim 2, wherein the cosmetic agent is for maintaining functioning of facial and body skin.

5. The agent of claim 3, wherein the therapeutic agent is for treatment of dermatosis.

6. A method for treating skin, the method comprising applying an agent to a skin area in need of treatment, the agent being an aqueous solution comprising bacteriorhodopsin from a halophilic bacterium Halobacterium salinarum VKPM V-11850 at a concentration of from 0.24 to 0.75% (wt/vol).

7. The method of claim 6, wherein the agent is a cosmetic agent.

8. The method of claim 6, wherein the agent is a therapeutic agent.

9. The method according to claim 7, wherein applying the cosmetic agent to the skin area is for maintaining functioning of the facial and body skin.

10. The method according to claim 9, wherein the cosmetic agent is applied once per day.

11. The method according to claim 8, wherein applying the therapeutic agent is for treatment of dermatosis.

12. The method according to claim 11, wherein the dermatosis is selected from the group consisting of psoriasis, eczema, neurodermatitis and atopic dermatitis.

13. The method according to claim 11, wherein the therapeutic agent is applied once or twice per day.
